(19) United States
(12) Patent Application Publication
(10) Pub. No.: US 2009/0286990 A1
(43) Pub. Date: Nov. 19, 2009

(54) PROCESS FOR PREPARING IRBESARTAN

(76) Inventors: Reguri Buchi Reddy, Hyderabad (IN); Sunkari Sudhakar, Hyderabad (IN); Chaganti Sridhar, West Godavari (IN); Somisetti Narender Rao, Nalgonda (IN); Sundaram Venkataraman, Hyderabad (IN); Padi Pratap Reddy, Hyderabad (IN); Bollikonda Satyanarayana, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 11/569,380

(22) PCT Filed: May 20, 2005
(86) PCT No.: PCT/US05/17953

§ 371 Date: Nov. 20, 2006

Related U.S. Application Data
(60) Provisional application No. 60/579,914, filed on Jun. 15, 2004.

(30) Foreign Application Priority Data
May 20, 2004 (IN) 472/CHE/2004

(51) Int. Cl. C07D 487/10; C07C 237/20

(52) U.S. Cl. 548/301.1; 564/155

(57) ABSTRACT

A process for preparing irbesartan comprises pentanoylation of cycloleucine in the presence of sodium hydroxide to form n-pentanoyl cycloleucine, condensing this product with 2-(4-aminomethyl phenyl) benzonitrile using dicyclohexyl carbodiimide and 1-hydroxy benzotriazole as a catalyst to form the 4-(&quest-N-pentanoyl amino) cyclopentamido methyl-2′-cyano biphenyl compound, and then cyclizing using trifluoroacetic acid in the presence of an aromatic solvent to form cyano irbesartan. Cyano irbesartan is converted to irbesartan by reaction with tributyltin chloride and sodium azide in the presence of an aromatic solvent.

The present invention relates to a process for preparing the compound 2-n-Butyl-3-[[2′-(1H-tetrazol-5-yl) [1,1′-biphenyl]-4-yl]methyl]-1,3-diazaspiro[4.4]non-1-en-4-one, also named 2-butyl-3[p-(o-1H-tetrazol-5-ylphenyl)benzyl]-1,3-diazaspiro[4,4]non-1-en-4-one, which is also known by the adopted name “irbesartan.” Pharmaceutical products containing irbesartan are being sold using the trademark AVAPRO, for treating hypertension. The compound can be represented by formula (I).

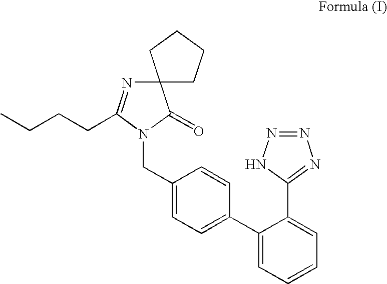

U.S. Pat. No. 5,352,788 disclosed and claimed certain N-substituted heterocyclic derivatives including 2-n-butyl-4-spirocylopentane-1-[(2-(tertrozol-5yl) biphenyl-4-yl)methyl]-2-imidazolin-5-one, commonly known as irbesartan, and pharmaceutical compositions containing them.

Irbesartan is a non-peptide compound, which antagonizes the physiologic effects of angiotensin II by inhibiting the action of angiotensin II on its receptors; the compounds particularly prevent increases in blood pressure produced by the receptor interaction. Thus, the compound Irbesartan is useful in the treatment of cardiovascular conditions such as hypertension and heart failure, as well as in preventing disorders of central nervous system, glaucoma, diabetic retinopathy, and diabetic nephropathy.

A process for preparing irbesartan has been described in U.S. Pat. Nos. 5,270,317 and 5,352,788. According to the patents, irbesartan can be prepared by reacting 2-n-butyl-4-spirocylopentane-2-imidazolin-5-one with 4-bromomethyl-2-cyanobiphenyl in the presence of NaOH, followed by a column chromatography separation to yield 1-[(2′-cyanobiphenyl-4-yl)methyl]-2-n-butyl-4-spirocyclopentane-2-imidazolin-5-one (II). This product compound is further reacted with tributyltin azide and the product treated with trityl chloride and separated by column chromatography. Finally, trityl protected irbesartan is deprotected with HCl and the final irbesartan product is isolated.

U.S. Pat. No. 6,162,922 has described a process for the preparation of 4-[[2-butyl-4-oxo-1,3-diazaspiro[4,4]non-1-ene-3yl]methyl][1,1-biphenyl]-2-carbonitrile (IV) comprising reacting 2-butyl-1,3-diazaspiro[4,4]nonane-4-one hydrochloride with 4′-(bromomethyl)[1,1-biphenyl]-2-carbonitrile (III) in the presence of phase transfer catalyst.

The known processes for preparing irbesartan involve tedious workup procedures, e.g., involve a large number of steps which include the protection and subsequent deprotection and isolation of intermediates, as well as separations by column chromatography. The processes of the art involve tedious workup to isolate the required product and this results in excessive production times, which in turn renders the process more costly and less eco-friendly; thus, the processes are not suitable for commercial scale-up. Accordingly, there remains a need for a simple, commercially advantageous process.

### SUMMARY OF THE INVENTION

In one embodiment, a process for preparing irbesartan comprises:

(a) reacting a compound having the formula III:

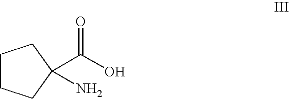

with valeryl chloride, to form a compound having the formula IV:

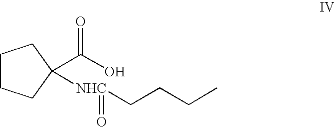

(b) reacting the compound having formula IV with a compound having formula V:

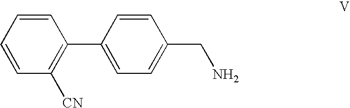

to form a compound having formula VI:

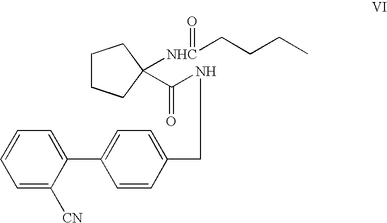

(c) cyclizing the compound having formula VI in the presence of an acid to form a compound having the formula II:

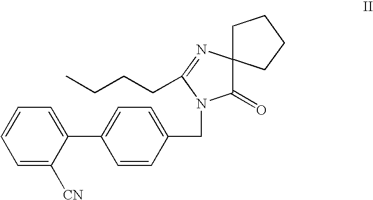

and

(d) reacting the compound having formula II with sodium azide to form irbesartan.

In a second embodiment, irbesartan is prepared by a process comprising:

(a) reacting a compound having formula VII:

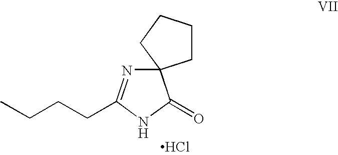

with 4-bromobenzylbromide to form a compound having formula IX:

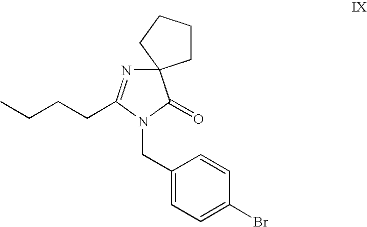

and;

(b) reacting the compound having formula IX with a compound having formula X:

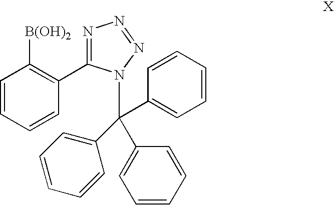

to form irbesartan.

### DETAILED DESCRIPTION

The present invention provides simple processes for preparing irbesartan. One embodiment of the present invention comprises pentanoylation of cycloleucine (III) in the presence of sodium hydroxide to form n-pentanoyl cycloleucine (IV), condensing this product with 2-(4-aminomethyl phenyl) benzonitrile (V) using dicyclohexyl carbodiimide and 1-hydroxy benzotriazole as a catalyst to form the 4-(α-N-pentanoyl amino) cyclopentamido methyl-2′-cyano biphenyl (VI) compound, and then cyclizing using trifluoroacetic acid in the presence of an aromatic solvent such as xylene or toluene to get cyano irbesartan (II). Cyano irbesartan is converted to irbesartan (I) by reaction with tributyltin chloride and sodium azide in the presence of an aromatic solvent such as toluene or xylene. Finally a pure pharma grade crystalline Form A irbesartan is isolated by recrystallization from methyl isobutyl ketone solvent.

The present invention relates to a novel and improved process for the preparation of 2-n-Butyl-3-[[2′-(1H-tetrazol-5-yl) [1,1′-biphenyl]-4-yl]methyl]-1,3-diazaspiro[4.4]non-1-en-4-one (commonly known as irbesartan). A process of the present invention is schematically represented as follows.

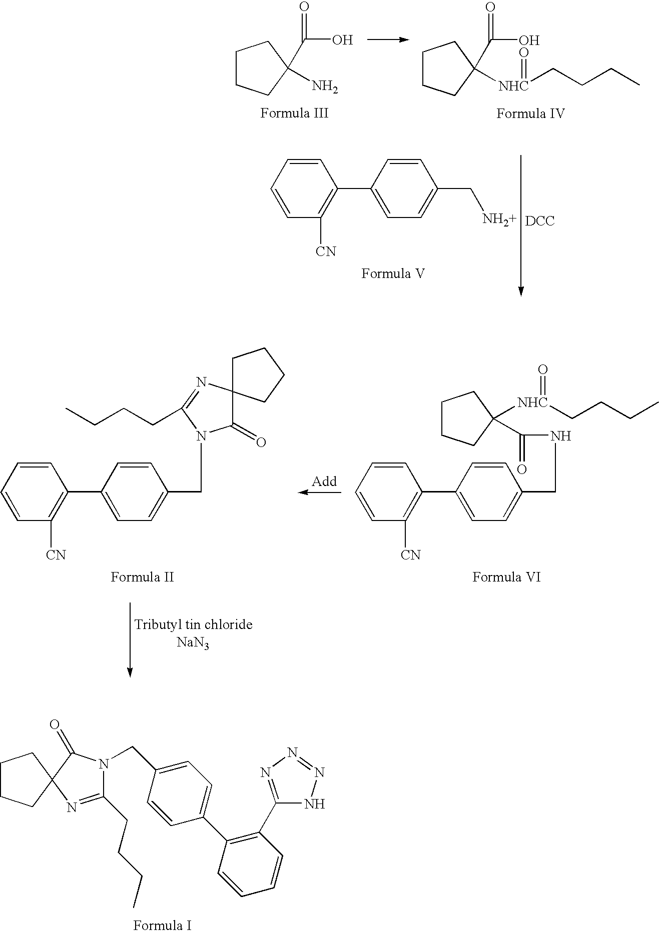

In one aspect, a process of the present invention comprises:

(1) reacting cycloleucine, or 1-amino-cyclopentanecarboxylic acid, (III) with valeryl chloride in the presence of an inorganic base such as sodium hydroxide, potassium hydroxide, sodium bicarbonate, potassium bicarbonate, sodium hydride, or organic bases such as triethyl amine, preferably sodium hydroxide, to produce n-pentanoyl cycloleucine, or 1-[(1-oxopentyl)amino]-cyclopentanecarboxylic acid, (IV);

(2) condensing the n-pentanoyl cycloleucine (IV) with 2-(4-aminomethyl phenyl) benzonitrile (V), using dicyclohexyl carbodimide and 1-hydroxy benzotriazole as a catalyst, in a solvent such as tetrahydrofuran or methylene chloride, to obtain 4-(α-N-pentanoyl amino) cyclopentamido methyl-2′-cyano biphenyl, or 1-(2′-cyanobiphenyl-4-ylmethylaminocarbonyl)-1-pentanoylaminocyclopentane, (VI);

(3) cyclization of the 4-(α-N-pentanoyl amino) cyclopentamido methyl-2′-cyano biphenyl (VI) in the presence of an acid such as trifluoroacetic acid, hydrochloric acid in isopropanol, or trichloroacetic acid, methanesulfonic acid, or phosphorous pentoxide and 4-methylbenzene sulfonic acid in a solvent selected from xylene or toluene or acetic acid or isopropanol or n-butanol and sec. butanol, to get cyano irbesartan, or 4′-[(2-butyl-4-oxo-1,3-diazaspiro[4,4]non-1-en-3-yl)methyl]-[1,1′-biphenyl]-2-carbonitrile, (II); and

(4) reacting the cyano irbesartan (II) with sodium azide in the presence of tributyl tin chloride or tributyl ammonium chloride in a solvent such as xylene, toluene, or a mixture thereof to get irbesartan (I).

If desired, a more pure pharmaceutical grade material can be obtained by recrystallizing the irbesartan from a solvent such as acetone, methyl ethyl ketone, methyl propyl ketone, methyl isobutyl ketone, acetonitrile, propionitrile, or mixtures of any two or more thereof.

The starting material cycloleucine, or 1-amino-1-cyclopentanecarboxylic acid, is commercially available and was described at Z. Physiol. Chem., Vol. 75, page 350 (1912). The compound 2-(4-aminomethyl phenyl) benzonitrile (V), or 4′-(aminomethyl)-[1,1′-biphenyl]-2-carbonitrile, has been described in U.S. Pat. No. 5,015,651.

Another aspect of the invention provides a process for the preparation of 2-n-butyl-3-[[2′-(1H-tetrazol-5-yl)[1,1′-biphenyl]4-yl]methyl]-1,3-diazaspiro[4.4]non-1-en-4-one of Formula (I), which is schematically depicted as follows:

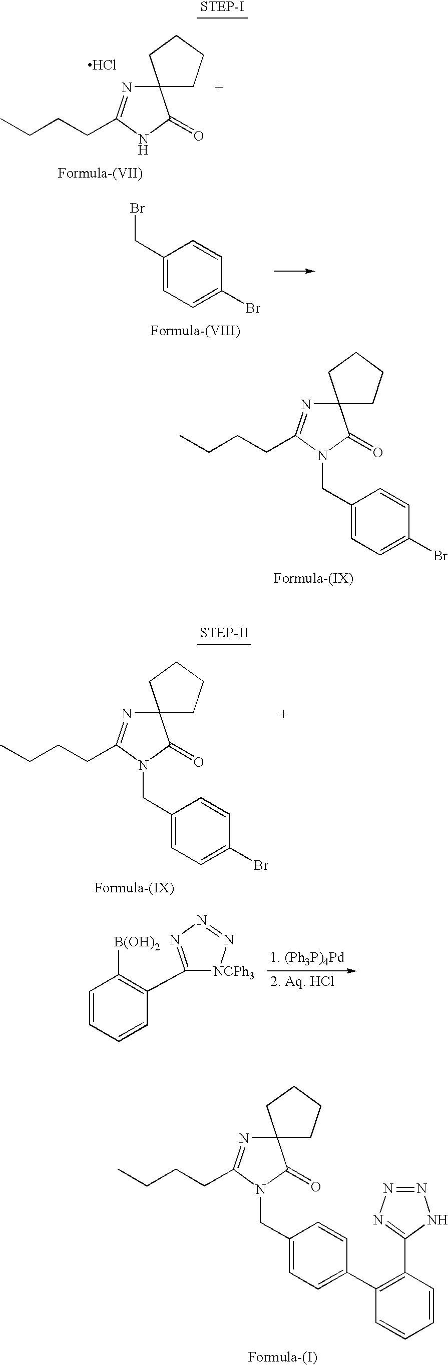

Accordingly, this process for the preparation of 2-n-butyl-3-[[2′-(1H-tetrazol-5-yl)[1,1′-biphenyl]-4-yl]methyl]-1,3-diazaspiro[4.4]non-1-en-4-one of Formula (I) comprises:

(a) reacting 2-Butyl-1,3-diazo spiro[4.4]non-1-en-4-one monohydrochloride of Formula (VII) with 4-bromobenzyl bromide of Formula (VIII) in a solvent such as toluene or dimethyl formamide, preferably dimethyl formamide, using a base such as potassium hydroxide, potassium carbonate, triethylamine, or sodium carbonate, preferably potassium carbonate, to yield 3-[4-bromo benzyl]-2-butyl-1,3-diazaspiro[4.4]non-1-en-4-one of Formula (IX);

(b) reacting 3-[4-bromo benzyl]-2-butyl-1,3-diazaspiro[4.4]non-1-en-4-one of Formula (IX) with 2-(2′-(triphenyl methyl-2′H-tetrazol-5′-yl) phenyl boronic acid under an inert atmosphere with tetrakis triphenyl phosphine palladium using a base such as potassium carbonate, sodium carbonate, or triethylamine preferably potassium carbonate, to afford irbesartan; and

(c) optionally, recrystallizing the compound obtained in step (b) using a ketone solvent such as methyl isobutyl ketone, methyl ethyl ketone, or methyl propyl ketone, preferably methyl isobutyl ketone, or alcohol solvents such as isopropanol, or nitrile solvents such as acetonitrile, to obtain recrystallized irbesartan.

The irbesartan prepared in the present simple process has crystalline Form A, with sufficient purity to be suitable for use in pharmaceutical formulations.

The following examples are only illustrative and are not intended to limit the scope of the invention as it is defined by the claims.

### Example 1

In a process for the preparation of n-pentanoyl cycloleucine of Formula IV, sodium hydroxide (68.2 grams, 1.7 moles) was added to water (275 ml) and cooled the solution cooled to 0-5° C., then there was added slowly a solution of cycloleucine (55 grams, 0.426 moles) and valeryl chloride (77 grams, 0.639 moles) in toluene (55 ml) over about 2-3 hours at 0-10° C. The reaction mass was maintained at 0-10° C. for about 2-3 hours. Water (275 ml) and toluene (55 ml) were added to the reaction mass and the mixture was stirred for about 15 minutes. The aqueous layer was separated and washed with toluene (55 ml), then the aqueous layer pH was adjusted to 2.0-2.5 with 8% aqueous hydrochloric acid (95 ml) and stirred for 15 minutes as a solid formed. The solid was separated by filtration and washed with water (45 ml), then was dried at 70-80° C. to get the desired compound of Formula IV, in an amount of 50 grams.

### Example 2

In a process for the preparation of 4-[(alpha-N-pentanoyl amino) cyclopentamido methyl]-2′-cyano biphenyl of Formula VI, a mixture of methylene chloride (750 ml), 4-amino-2′-cyano 1,1′-biphenyl of Formula V (30 grams, 0.144 moles), n-pentanoyl cycloleucine of Formula IV (33 grams, 0.158 moles), hydroxy benzotriazole (3.9 grams, 0.028 moles) and dicyclohexyl carbodiimides (29.7 grams, 0.144 moles) was stirred at 25-35° C., until the reaction was complete. The formed solid was filtered and washed with methylene chloride (30 ml) followed by washing the filtrate with saturated sodium bicarbonate solution (2×250 ml). The organic layer was separated and concentrated under reduced pressure. Cyclohexane (100 ml) was added to the residue and the mixture stirred for 15 minutes. Then the separated solid was removed by filtration, washed with cyclohexane (30 ml), and dried at 70-80° C. to a constant weight to yield the desired compound (59 grams).

### Example 3

A process for the preparation of 1-[(2′-Cyanobiphenyl-4-yl) methyl]-2-n-butyl-4-spirocyclopentane-2-imidazolin-5-one of Formula (II) involved charging 4-[(alpha-N-pentanoyl amino) cyclopentamido methyl]-2′-cyano biphenyl (140 ml, 0.347 moles) toluene (1400 ml) and trifluoroacetic acid (40.1 ml) to a vessel, heating to reflux temperature and maintaining until completion of the reaction. After cooling the reaction mass to 25-35° C., the mixture was washed with water (1×400 ml and 1×280 ml) and the organic layer was separated. To the organic layer was added 6% aqueous HCl (1120 ml) and the mixture was stirred for 1-2 hours. The mixture was further cooled to 0-5° C. and stirred for 2 hours. The solid was filtered, washed with toluene (140.0 ml), and dried at 70-80° C. to get the desired compound (88.0 grams).

### Example 4

In a process for the preparation of 2-Butyl-3-[[2′-(1H-tetrazol-5-yl) [1,1′-biphenyl]-4-yl]methyl]-1,3-diazaspiro[4.4]non-1-en-4-one (I), a solution of 1-[(2′-cyanobiphenyl-4-yl) methyl]-2-n-butyl-4-spirocyclopentane-2-imidazolin-5-one hydrochloride (50 grams, 0.1186 moles) and toluene (250 ml) was charged into a mixture of 17% liquid ammonia (29.6 ml, 0.296 moles) and water (500 ml). The reaction mass was stirred at 30-35° C. for 35-45 minutes, then filtered and the solid washed with toluene (150 ml). The aqueous layer was separated and extracted with toluene (150 ml). The organic layers were combined and concentrated under reduced pressure. Xylene (50 ml) was added to the residue followed by tributyltin chloride (77.1 grams, 0.237 moles) and sodium azide (15.4 grams, 0.2369 moles), and the reaction mass heated to reflux until the reaction completed. The reaction mass was cooled to 25-35° C. and water (500 ml) and acetone (400 ml) were added. Reaction mass pH was adjusted to 4.0 to 4.5 with a 1:3 solution of acetic acid and water (140 ml), then cyclohexane (500 ml) was added and the mixture stirred for 2 hours. A solid was isolated by filtering the reaction mass and was washed with cyclohexane (250 ml). After drying the solid at 70-80° C. the desired compound was obtained (48 grams).

### Example 5

Irbesartan was purified by recrystallization from methyl isobutyl ketone. A mixture of irbesartan (11 grams) and methyl isobutyl ketone (330.0 ml) was heated to reflux. Decolorizing carbon powder was added to the reaction solution and maintained at reflux for 35-45 minutes. Carbon was removed by filtering the mixture in a hot condition and washing the carbon with methyl isobutyl ketone (11 ml). The filtrate was cooled to 25-30° C. and stirred for 45 minutes and then further cooled to 0-5° C. and stirred for 45 minutes to produce a solid. The solid was isolated by filtration and washed with methyl isobutyl ketone (11 ml). After drying at 70-80° C., the desired compound was obtained (8.8 grams).

### Example 6

Irbesartan was purified by recrystallization from isopropyl alcohol. Irbesartan (30 grams) and isopropyl alcohol (600 ml) were mixed and heated to reflux. After 15 minutes, the solution was filtered and the solid washed with isopropyl alcohol (30 ml). The filtrate was cooled to 20-25° C. with stirring for 30 minutes to produce a solid. The solid was isolated by filtration and washed with water (60 ml). The obtained solid was dried at 70-80° C. to yield the desired compound (20.9 grams).

### Example 7

3-[4-bromo benzyl]-2-butyl-1,3-diazaspiro[4.4]non-1-en-4-one was prepared by adding a mixture of 2-Butyl-1,3-diazo spiro[4.4]non-1-en-4-one Hydrochloride (50 grams), potassium carbonate (74.8 grams), 4-bromobenzyl bromide (67.8 grams) at a temperature of 25-35° C. to dimethylformamide (400 ml), heating to a temperature of 50-55° C., and then stirring gently and maintaining at temperature until TLC analysis indicated reaction completion. Subsequently, the reaction mixture was cooled to a temperature of 25-35° C. A mixture of toluene (500 ml) and water (800 ml) was added to the reaction mixture and stirred for 45-60 minutes, then the organic layer was separated and washed with water (6×400 ml). Solvent was distilled off to obtain the desired compound. (Yield: 60.2 grams, 76%).

### Example 8

The preparation of Irbesartan is accomplished by adding a mixture of 3-[4-Bromo benzyl]-2-butyl-1,3-diazaspiro[4.4]non-1-en-4-one (5.0 grams, prepared according to preceding Example 7), 2-(2′-Triphenylmethyl-2′H-tetrazol-5′-yl)-phenylboronic acid (6.1 grams, prepared according to Example 1 of U.S. Pat. No. 5,310,928), and potassium carbonate (4.7 grams) to toluene (60 ml) followed by water addition (0.7 ml) under a nitrogen atmosphere. Tetrakis(triphenyl phosphine)palladium (0.4 grams) was added to the above reaction mixture at a temperature of 25-35° C., and the reaction mixture was heated to a temperature of 80-85° C. and stirred until completion of the reaction as shown by TLC analysis. The reaction mixture was cooled to a temperature of 25-35° C. and water (50 ml) was added and stirred for 30 minutes at a temperature of 25-35° C. The resultant reaction mixture was filtered and washed with toluene (20 ml), then the organic layer was separated and washed with water (20 ml). Solvent was distilled from the organic layer under reduced pressure. The resulting residue was dissolved in dichloromethane (50 ml) and washed with saturated sodium chloride solution (2×20 ml). Solvent was distilled from the organic layer. Methyl isobutyl ketone (50 ml) was added to the resulting residue followed by addition of 30% aqueous hydrochloric acid (10 ml) and stirring until the reaction was complete, as shown by TLC analysis. Solvent was distilled from the reaction mixture under a reduced pressure. Sodium hydroxide (3 grams) in water (30 ml) was added followed by water (100 ml) and the mixture was stirred for 30 minutes. The aqueous layer was washed with toluene (3×15 ml) at a temperature of 10-15° C., then the aqueous layer was separated and its pH was adjusted to 5 using dilute acetic acid. Upon stirring for 1-2 hours at a temperature of 25-35° C. the produced solid mass was filtered and washed with water (25 ml) to afford the desired compound. (Yield: 1.3 grams, 22%).

1-11. (canceled)

12. A process for preparing irbesartan, comprising:
cyclizing a compound having Formula VI: 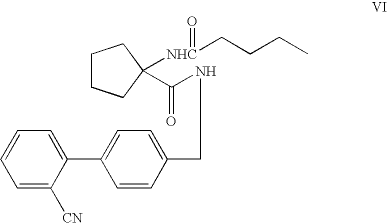
in the presence of an acid to form a compound having Formula II:

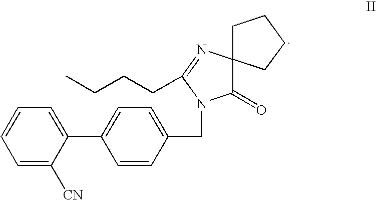

13. The process of claim 12, wherein an acid comprises trifluoroacetic acid, hydrochloric acid in isopropanol, trichloroacetic acid, methanesulfonic acid, phosphorous pentoxide, or 4-methylbenzene sulfonic acid.

14. The process of claim 12, wherein an acid comprises trifluoroacetic acid.

15. The process of claim 12, wherein cyclizing is carried out in the presence of a solvent comprising toluene, xylene, acetic acid, isopropanol, n-butanol, or sec-butanol.

16. The process of claim 12, wherein cyclizing is carried out in the presence of a solvent comprising toluene.

17. The process of claim 12, further comprising reacting with an azide to form irbesartan.

18. The process of claim 12, further comprising reacting with sodium azide to form irbesartan.

19. The process of claim 17, wherein reacting with an azide is conducted in the presence of tributyl tin chloride or tributyl ammonium chloride; in a solvent selected from xylene and toluene.

20. The process of claim 17, wherein reacting with an azide is conducted in the presence of tributyl tin chloride or tributyl ammonium chloride, in a solvent comprising xylene or toluene.

21. The process of claim 17, further comprising recrystallizing irbesartan from a solvent.

22. The process of claim 21, wherein a solvent comprises a ketone, an alcohol, a nitrile, or a mixture of any two or more thereof.

23. The process of claim 21, wherein a solvent comprises isopropanol.

24. The process of claim 17, wherein irbesartan has crystalline form A.

25. The process of claim 12, wherein a compound having Formula VI: 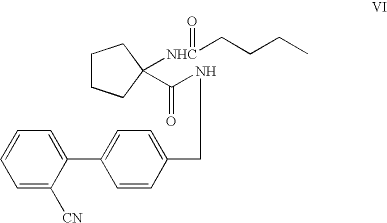
is prepared by a process comprising:
reacting a compound having Formula III: 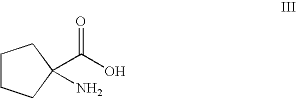
with valeryl chloride, to form a compound having Formula IV: 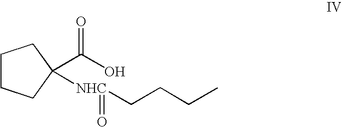
and further reacting with a compound having Formula V:

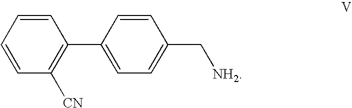

26. The process of claim 25, wherein a reaction with valeryl chloride occurs in the presence of a base.

27. The process of claim 25, wherein a reaction with valeryl chloride occurs in the presence of an inorganic base.

28. A process for preparing irbesartan, comprising:
reacting a compound having Formula VIII: 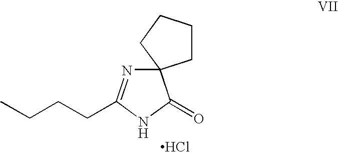
with 4-bromobenzylbromide to form a compound having Formula IX: 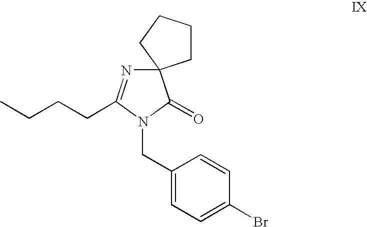
and further reacting with a compound having Formula X:

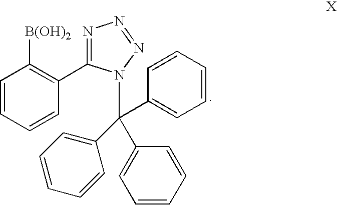

29. The process of claim 28, wherein a reaction with 4-bromobenzylbromide occurs in the presence of a base.

30. The process of claim 28, wherein a reaction with a compound having Formula X occurs in the presence of tetrakis(triphenylphosphine)palladium.

31. The process of claim 28, wherein a reaction with 4-bromobenzylbromide occurs in the presence of a base, and a reaction with a compound having Formula X occurs in the presence of tetrakis(triphenylphosphine)palladium.
